(19) United States
(12) Patent Application Publication
(10) Pub. No.: US 2023/0161998 A1
(43) Pub. Date: May 25, 2023

(54) SYSTEMS AND METHODS FOR PROCESSING ELECTRONIC IMAGES TO DETERMINE ONCOGENIC SIGNALS

(71) Applicant: PAIGE.AI, Inc., New York, NY (US)

(72) Inventors: Yikan WANG, Burnaby (CA); Jeremy KUNZ, New York, NY (US); Christopher KANAN, Pittsford, NY (US)

(21) Appl. No.: 18/051,304

(22) Filed: Oct. 31, 2022

Related U.S. Application Data
(60) Provisional application No. 63/264,465, filed on Nov. 23, 2021.

(51) Int. Cl. G06N 3/04; G06N 3/08

(52) U.S. Cl. CPC G06N 3/04; G06N 3/08

(57) ABSTRACT

Systems and methods are disclosed for generating and predicting behavior of patient-specific oncogenic signaling pathways or networks. In some aspects, patient-specific oncogenic signaling pathways or networks may be generated by receiving one or more digital medical images associated with a patient, providing an unpopulated gene network graph and the one or more digital medical images as input to a trained machine learning system that is trained to populate the gene network graph with gene expression levels specific to the patient based on the one or more digital medical images, and receiving, as output from the trained machine learning system, the gene network graph populated with the gene expression levels specific to the patient.

### CROSS-REFERENCE TO RELATED APPLICATIONS

This patent application claims the benefit of U.S. Provisional Application No. 63/264,465, filed on Nov. 23, 2021, the entirety of which is incorporated by reference herein.

### FIELD OF THE DISCLOSURE

Various techniques of the present disclosure pertain generally to oncogenic signaling pathway analysis. More specifically, particular techniques of the present disclosure relate to systems and methods for generating and predicting behavior of patient-specific oncogenic signaling pathways or networks.

### BACKGROUND

A signaling pathway, also referred to as a biochemical cascade, is a series of chemical reactions that occur within a biological cell when initiated by a stimulus. For example, a signaling pathway for a given cancer may include one or more mutated genes and the aberrant molecules generated by said genes. Generally, signaling pathways are depicted using a gene network graph. The gene network graph may include nodes that depict a gene with attached expression level to graphically depict the signaling pathway. Gene network graphs may be used to show genetic alterations in signaling pathways that control cell cycle progression, apoptosis, and cell growth, which are hallmarks of cancer. Identification of actionable alterations in the genetic signaling pathways suggest opportunities for targeted and combination therapies to treat cancer.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for generating and predicting behavior of patient-specific oncogenic signaling pathways or networks. Each of the aspects of the disclosure herein may include one or more of the features described in connection with any of the other disclosed aspects.

According to one example of the present disclosure, methods for processing digital medical images to populate gene network graphs, or a data representation of a gene network graph, may be described. An exemplary method may include receiving one or more digital medical images associated with a patient; providing an unpopulated gene network graph and the one or more digital medical images as input to a trained machine learning system that is trained to populate the gene network graph with gene expression levels specific to the patient based on the one or more digital medical images; and receiving, as output from the trained machine learning system, the gene network graph populated with the gene expression levels specific to the patient.

According to another example of the present disclosure, methods for training a machine learning system to populate a gene network graph may be described. An exemplary method may include receiving an unpopulated gene network graph, the unpopulated gene network graph comprising a gene network graph without expression levels; receiving tumor sequencing information associated with each of a plurality of patients; receiving one or more digital medical images associated with each of the plurality of patients; populating, for each of the plurality of patients, the gene network graph to include expression levels based on the respective tumor sequencing information; and training the machine learning system to infer one or more of the populated gene network graphs based on the respective one or more digital medical images.

According to a further example of the present disclosure, systems for processing digital medical images to populate gene network graphs may be described. An exemplary system may include at least one memory storing instructions and at least one processor configured to execute the instructions to perform operations. The operations may include receiving one or more digital medical images associated with a patient; providing an unpopulated gene network graph and the one or more digital medical images as input to a trained machine learning system that is trained to populate the gene network graph with gene expression levels specific to the patient based on the one or more digital medical images; and receiving, as output from the trained machine learning system, the gene network graph populated with the gene expression levels specific to the patient.

It is to be understood that both the foregoing general description and the following detailed description are exemplary and explanatory only and are not restrictive of the disclosed embodiments, as claimed.

### BRIEF DESCRIPTION OF THE FIGURES

The accompanying drawings, which are incorporated in and constitute a part of this specification, illustrate various exemplary techniques and together with the description, serve to explain the principles of the disclosed techniques.

FIG. 1A depicts a block diagram of an exemplary system for populating and predicting changes in gene network graphs, according to one or more techniques.

FIG. 1B depicts a block diagram of an exemplary system for populating gene network graphs, according to one or more techniques.

FIG. 1C depicts a block diagram of an exemplary system for predicting changes in gene network graphs, according to one or more techniques.

FIG. 1D depicts an exemplary gene network graph, according to one or more techniques.

FIG. 2 depicts a schematic of an exemplary system for populating a gene network graph, according to one or more techniques.

FIG. 3 depicts a schematic of an exemplary system for predicting changes to a gene network graph, according to one or more techniques.

FIG. 4 depicts a flow diagram for an exemplary process for generating and predicting changes in gene network graphs, according to one or more techniques.

FIG. 5 depicts a flow chart of an exemplary method for populating gene network graphs, according to one or more techniques.

FIG. 6 depicts an exemplary method for training a machine learning model for a graph generation system, according to one or more techniques.

FIG. 7 depicts a flow chart of an exemplary method for predicting gene network graph changes, according to one or more techniques.

FIG. 8 depicts an exemplary method for training a machine learning model for a graph prediction system, according to one or more techniques.

FIG. 9 illustrates an example system or device that may execute techniques presented herein, according to one or more techniques.

### DETAILED DESCRIPTION

Reference will now be made in detail to the exemplary techniques of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

As used herein, the term “gene expression” refers to the process by which information from a gene is used in the synthesis of a functional gene product that enables the gene to produce end products, such as protein or non-coding RNA, and ultimately affect a phenotype, as the final effect. Regulation of gene expression is the control of the amount and timing of appearance of the functional product of a gene. Control of expression allows a cell to produce the gene products the cell needs when the cell needs them; in turn, this gives the cell the flexibility to adapt to a variable environment, external signals, damage to the cell, and/or other stimuli. A gene (or genetic) regulatory network (GRN) is a collection of molecular regulators that interact with each other and with other substances in the cell to govern the gene expression levels of mRNA and proteins which, in turn, determine the function of the cell.

As used herein, the term “gene network signaling graph,” “gene network graph,” or the like may be a weighted, directed graph comprised of nodes and edges connecting the nodes, or data structure representing a gene network graph. Each node may be a gene with an attached expression level that can be either continuous or thresholded to be discrete. The edges of the graph may define the signaling pathway graph based on known findings in genetics. The continuous expression levels (continuous weights) indicate the level of activity for a gene. In some examples, values of the continuous weights may be thresholded to simplify analysis. In such examples, thresholds may be obtained from results of scientific studies aimed at determining when an expression level is aberrant. As used herein, the term “populated gene network graph” or the like may be a gene network graph, or data structure representing a gene network graph, that is populated with gene expression levels. As used herein, the term “unpopulated gene network graph” or the like may be a gene network graph that is not populated with gene expression levels. For example, the unpopulated gene network graph may include a structure, configuration, or topology of the nodes and edges depicting interactions between genes, but does not include patient-specific expression levels associated with the genes.

As used herein, “driver mutations,” “driver genetic alterations,” “driver epigenetic alterations,” and the like refer to genetic mutations that drive the development of cancer. Driver mutations are mutations that allow cancer to proliferate and invade human cells, e.g., somatic cells.

Epigenetic changes, such as abnormal patterns of DNA acetylation and/or methylation, disrupted patterns of histone posttranslational modification, and/or chromatin remodeling, may cooperate with genetic alterations to generate a cancer phenotype. For example, epigenetic alterations as an alternative driver of carcinogenesis may cause mutations in genes and, conversely, mutations may be frequently observed in genes that modify the epigenome. There may be an association between genetic and/or epigenetic pathways (such as microsatellite instability, chromosomal instability, and promoter hypermethylation), and patient prognosis, overall survival, and/or response to targeted treatments in cancers. Therefore, genetic alterations and potentially reversible epigenetic changes in oncogenic signaling pathways may be used to inform therapeutic options for precision medicine.

For example, there have been successful treatments developed based on small molecular inhibitors like anti-HER2 drugs in HER2+ breast cancer. However, toxic chemotherapy and inevitable resistance to targeted therapy agents remain challenging in cancer treatment. Driver genetic and epigenetic alterations that predict response to targeting drugs may result in a specific histologic phenotype identifiable by microscopy. Therefore, artificial intelligence (AI) systems may be used to predict the presence of predictive biomarkers in whole-slide imaging (WSI) of tumor samples, and be used as screening tools for therapy decision making. However, one of the challenges to establish such AI systems may be due to lack of data and the low prevalence of individual genetic alterations in a given tumor type.

Additionally, although deep learning may be implemented to make predictions about oncogenic mutations in individual genes from digital histology imagery, reliance on predictions associated with only individual genes may be an over-simplification. A tumor's growth is governed by not just one gene, but many (or all) genes interacting in a network where one gene influences the activity of other genes. For example, different genetic and epigenetic alterations in multiple genes may result in perturbation of the same signaling pathway and a convergent phenotype. Genetic alterations and epigenetic aberrations may be considered an alternative driver of carcinogenesis, but the extent, mechanisms, and co-occurrence of alterations in their oncogenic pathways differ across different tumor types and individual tumor samples. Therefore, uncovering the effect of oncogenic signaling pathways perturbation to promote a cancer phenotype may involve integration of multi-layer evidence of genetic, epigenetic, and clinical presentation.

Techniques discussed herein may use AI technology, machine learning, and/or image processing tools applied to databases of patient histology images, patient clinical information, genomic data, and/or gene network relationships, among other data types, to generate and predict the behavior of patient-specific oncogenic signaling pathways and networks, e.g., gene network graphs. For example, a system may be established that generates a patient-specific gene network graph populated with corresponding levels of activity for each gene (e.g., representing a signaling pathway). Additionally, the system may detect changes at the signaling pathway level from digital medical images (e.g., histological WSIs) by integrating data from multiple sources including genomic variants and epigenetic alterations, signaling pathways, clinical presentation, and treatment outcomes. For examples, the system may identify computational and/or learned histological features associated with aberrations in oncogenic signaling pathways or complexes, and predict which oncogenic signaling pathways are driving patient tumor development, which may be exploited as therapeutic targets.

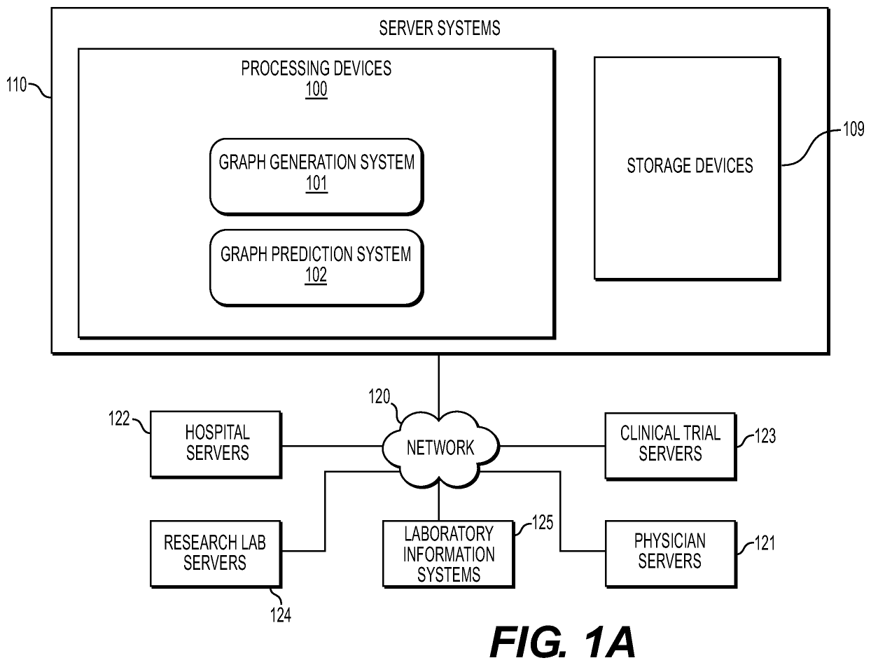

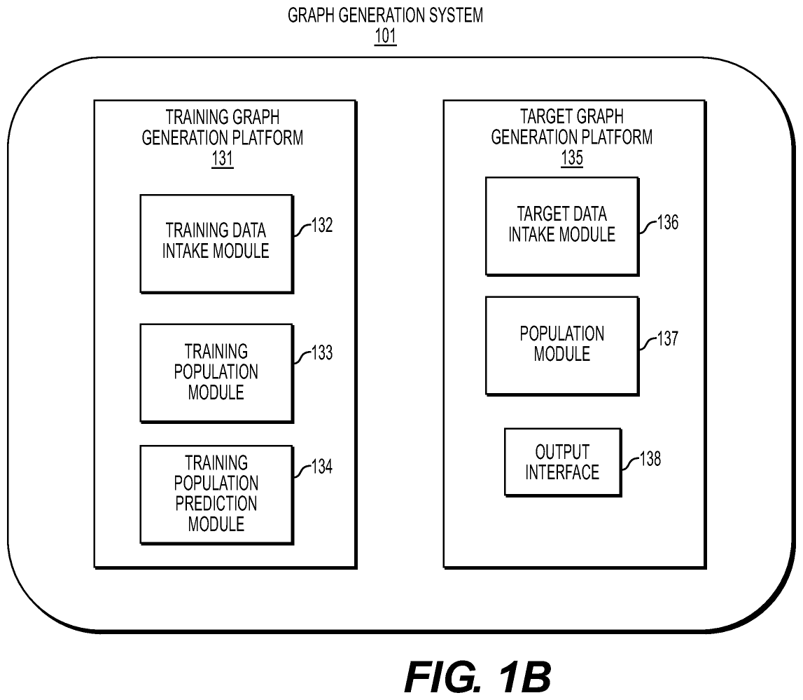

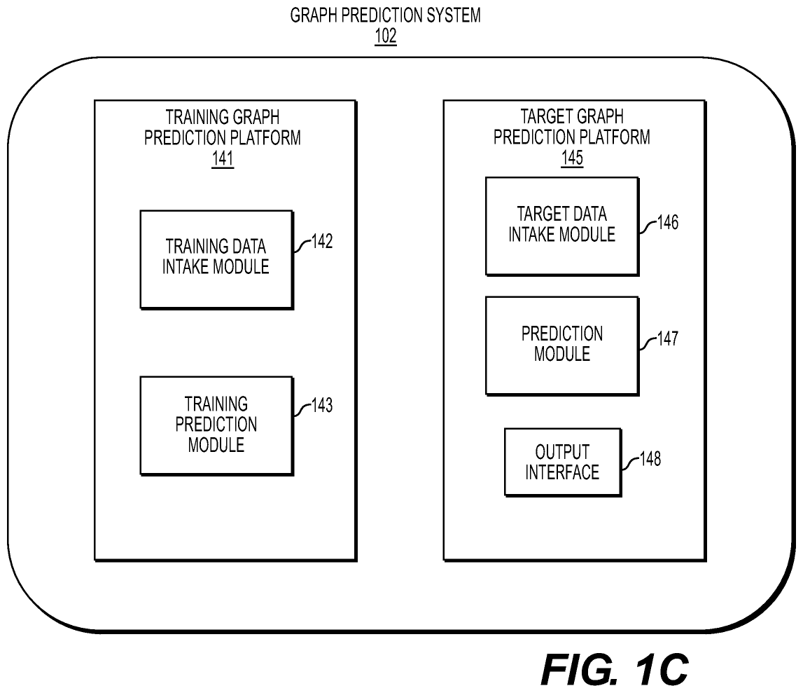

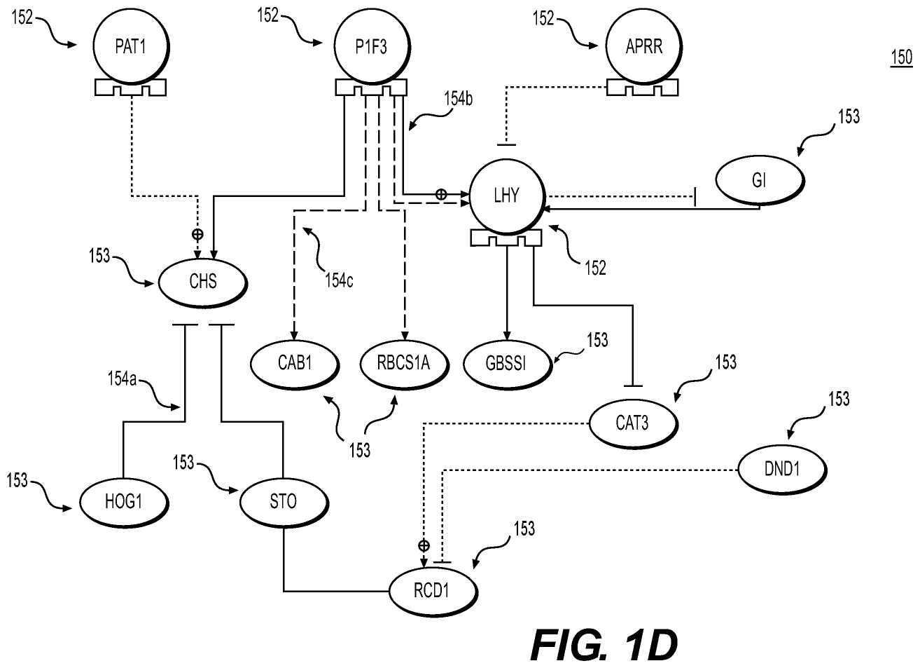

FIG. 1A depicts an exemplary system for generating and predicting behavior of patient-specific oncogenic signaling pathways or networks, according to one or more techniques. Illustrated in FIG. 1A is an electronic network 120 that may be connected to physician servers 121, hospital servers 122, clinical trial servers 123, research lab servers 124, and/or laboratory information systems 125, for example, through one or more computers, servers, and/or handheld mobile devices. According to an exemplary aspect of the present disclosure, network 120 may be connected to server systems 110, which may include one or more processing devices 100, e.g., configured to run or execute graph generation system 101 and graph prediction system 102, and/or storage devices 109. Graph generation system 101 may be configured for populating gene network graphs using one or more trained machine learning systems. Graph prediction system 102 may be configured for predicting behavior of gene network graphs, e.g., populated gene network graphs generated by the graph generation system 101 or another system, using one or more trained machine learning systems, according to exemplary aspects of the present disclosure. While graph generation system 101 and graph prediction system 102 are depicted as separate systems in FIG. 1A, it should be understood that, in other examples, graph generation system 101 and graph prediction system 102 may be sub-systems of a larger system.

Physician servers 121, hospital servers 122, clinical trial servers 123, research lab servers 124, and/or laboratory information systems 125 may create or otherwise obtain data, such as digital medical images, expression data, genomic variants, and/or clinical data. For example, the digital medical images may include digital pathology images, including one or more patients' whole slide image(s), cytology specimen(s), histopathology specimen(s), slide(s) of the cytology specimen(s), digitized images of the slide(s) of the histopathology specimen(s), or any combination thereof, that may be created or obtained. Additionally or alternatively, the digital medical images may include images of other modality types, including digital multiplex immunofluorescent images, digital multiplex immunohistochemistry images, magnetic resonance imaging (MRI), computed tomography (CT), X-ray, nuclear medicine imaging, or ultrasound, that may be created or obtained.

Expression data may include patient-specific or non-patient-specific tumor sequencing data, protein expression levels, and/or non-coding RNA expression levels. Expression data may be utilized by both medical professionals (e.g., pathologists, physicians, etc.) and AI systems alike for training purposes to improve accuracy in predicting oncogenic patterns, among other tasks. A greater availability of expression data presenting a particular condition or disease enhances both medical professionals' and AI systems' ability to learn given the increased variability in the presentation among the expression data. However, large amounts of expression data remain unavailable for individual genetic mutations in a given tumor type, which necessarily limits an amount of variability that can be learned. For example, treatment of a patient-specific tumor may be made difficult due to genotype variance compared to another patient with the same phenotype but a different genotype.

Genomic variants may include mutations in individual genes of a given gene complex or signaling pathway, such as the SWI/SNF complex (e.g., ARID1A, ARID1B, ARID2, PBRM1, SMARCA4 and SMARCB1) or the RTK/RAS pathway (e.g., ERBB2, ERBB3, ERBB4, SOS1, HRAS, BRAF, MAP2K1, and MAPK1), etc. Clinical data may include age, medical history, cancer treatment history, family history, past biopsy or cytology information, tumor sequencing information, mRNA expression levels, gene network graphs (pre-treatment and/or post-treatment), overall survival data, progression-free survival with corresponding censored data, 5-year survival rates, drug treatment outcome data, etc.

Digital medical images, expression data, genomic variants, clinical data, and/or other data may be communicated between server systems 110 and physician servers 121, hospital servers 122, clinical trial servers 123, research lab servers 124, and/or laboratory information systems 125 over network 120 in a digital or electronic format.

Server systems 110 may include one or more storage devices 109 for storing data, e.g., digital medical images, expression data, genomic variants, clinical data, received from at least one of physician servers 121, hospital servers 122, clinical trial servers 123, research lab servers 124, and/or laboratory information systems 125. For example, the one or more populated gene network graphs generated by graph generation system 101 may be stored within the one or more data stores, e.g., storage devices 109.

Server systems 110 may include processing devices 100 for processing the digital medical images and/or other above-described data stored in storage devices 109. Server systems 110 may include one or more machine learning tool(s) or capabilities. For example, processing devices 100 may execute one or more machine learning systems utilized by graph generation system 101 and/or graph prediction system 102, according to one or more techniques. In some examples, outputs of the machine learning systems may be stored in storage devices 109 for use by other systems or processes, as described in detail below. Alternatively or in addition, the present disclosure (or portions of the system and methods of the present disclosure) may be performed on a local processing device (e.g., a laptop).

According to an exemplary aspect of the present disclosure, graph generation system 101 may be configured to populate a gene network graph using one or more machine learning systems. The populated gene network graph may be patient specific and may include the corresponding levels of activity for each gene. According to an exemplary aspect of the present disclosure, graph prediction system 102 may be configured to predict how a populated gene network graph may behave over time with or without one or more treatments, using one or more machine learning systems. This implementation may make patient-specific data available to more accurately predict oncogenic changes, e.g., gene expression changes, in response to particular treatments.

FIG. 1B depicts an exemplary system (e.g., graph generation system 101) for generating populated gene network graphs, according to an exemplary aspect of the present disclosure. The graph generation system 101 may include a training graph generation platform 131 and/or a target graph generation platform 135.

The training graph generation platform 131, according to one technique, may create or receive one or more datasets of training data used to generate and train one or more machine learning models that, when implemented, populate a gene network graph with gene expression level and/or predicted tumor gene expression levels. According to one technique, the training graph generation platform 131 may include a plurality of software modules, including a training data intake module 132, a training population module 133, and a training population prediction model 134. The data and/or machine learning systems output by training graph generation platform 131 may be stored, e.g., in storage device 109, or used by other systems, e.g., target graph generation platform 135.

Training data intake module 132, according to one aspect, may create or receive training data (e.g., unpopulated gene network graphs, expression data, digital medical images, optional clinical data, etc.) that may be used to train one or more machine learning systems to generate populated gene network graphs. The training data may be received from any one or any combination of server systems 110, physician servers 121, hospital servers 122, clinical trial servers 123, research lab servers 124, and/or laboratory information systems 125. Training data may come from real sources (e.g., humans, animals, etc.) or may come from synthetic sources (e.g., graphics simulators, graphics rendering engines, 3D models, etc.).

The training data datasets may include one or more datasets corresponding to unpopulated gene network graphs, one or more datasets corresponding to expression data (e.g., RNA expression data), one or more datasets corresponding to tumor sequencing information, one or more datasets corresponding to digital medical images, and/or one or more datasets corresponding to clinical data. In some examples, a subset of training data may overlap between or among the various datasets for gene network graphs, tumor sequencing information, expression data, and/or clinical data. The training datasets may be stored on a digital storage device, e.g., one of storages devices 109.

In some examples, expression data, e.g., gene expression data and/or RNA expression data, may be a direct output of one or more of the machine learning systems. In other examples, the output of one or more of the machine learning systems may be used as input to further processes that enable generation of the populated gene network graphs. In another example, training WSI may include digitized histology or cytology slides stained with a variety of stains, such as, but not limited to, Hematoxylin and eosin, hematoxylin alone, toluidine blue, alcian blue, Giemsa, trichrome, acid-fast, Nissl stain, etc. Other training data may include genomic variants, and/or clinical data, as discussed herein. Clinical data may include histological data, tumor subtype data, tumor grading or staging data, tumor sizing data, patient demographic data, etc.

The training population module 133 may populate a gene network graph based at least on the tumor sequencing information. The unpopulated gene network graph may be generated by one or more systems, e.g., training population module 133, based on interaction data associated with a given set of genes obtained from a published study or other similar information source. Additionally or alternatively, the unpopulated gene network graph may be received from a public database that stores a collection of unpopulated gene network graphs for various sets of genes (e.g., the unpopulated gene network may be pre-made and stored in the public database by a third party and provided as input to the training population module 133). The tumor sequencing information may be received from any one or any combination of server systems 110, physician servers 121, hospital servers 122, clinical trial servers 123, research lab servers 124, laboratory information systems 125, and/or training data intake module 132. The training population module 133 may output, e.g., a gene network graph populated with patient-specific expression levels, that may be stored, e.g., in storage devices 109, and/or utilized by training population prediction module 134 in training.

In some examples, the tumor sequencing information may be obtained from a gene panel. However, a gene panel may not acquire expression levels from all genes, but only a subset of genes. Thus, the tumor sequencing information may be incomplete and may not be able to fully populate the gene network graph. In such instances, the genes that are not included in the gene panel may be treated as missing values and determined using, e.g., a label propagation algorithm (LPA) to enable the gene network graph to be fully populated. For example, the inference of the missing values corresponding to the expression level of the unanalyzed genes may be performed using a directed LPA.

In addition to populating gene network graph using the tumor sequencing information, training population module 133 may be configured correlate patient-specific tumor sequencing data with a phenotype, e.g., expression of a given onco-regulatory gene with tumor expression, and/or add tumor sequencing and gene expression data to a database, e.g., storage devices 109, of other sequencing and expression data. In some examples, a third party may train one or more of the machine learning systems of training population module 133 and provide the trained machine learning system(s) to server systems 110 for storage (e.g., in storage devices 109) and execution, e.g., by target graph generation platform 135.

Training population prediction module 134 may be trained to infer the populated gene network graph from digital medical images. In other words, training population module 133 may be configured to predict a populated gene network graph generated based on the gene expression data associated with digital medical images for a given tumor, e.g., how a tumor with a given phenotype will interact with other genes. In some examples, training population prediction module 134 may be further trained using clinical data, such as overall survival data, progression-free survival with corresponding censored data, drug treatment outcome data, etc. Exemplary methods for training the one or more machine learning systems of training population prediction module 134 are discussed in detail below.

In some examples, a machine learning system may be generated for each of the different tissue and/or tumor types to learn a corresponding gene network graph. In other examples, one machine learning system may be generated that is capable of learning gene network graphs for more than one tissue and/or tumor type. Training population prediction module 134 may generate one or more machine learning systems that are configured to operate via any of a multi-modal deep neural network, a graph neural networks, a convolutional neural network, a transformer neural network, etc.

According to one technique, the target graph generation platform 135 may include software modules, such as a target data intake module 136, a population module 137, and an output interface 138. Target graph generation platform 135, according to one aspect, may receive a request for expression data and execute one or more of the machine learning systems trained by training graph generation platform 131 to generate one or more populated gene network graphs. For example, the request may be received from any one or any combination of the physician servers 121, hospital servers 122, clinical trial servers 123, research lab servers 124, and/or laboratory information systems 125. In another example, the request may be automatically received from graph prediction system 102 in response to graph prediction system 102 receiving a request to predict a gene network graph and/or receiving a patient-specific unpopulated gene network graph.

Target data intake module 136, according to one aspect, may create or receive target data (e.g., images, optionally clinical data, etc.) that may be used as an input for one or trained more machine learning systems to generate populated gene network graphs. For example, target data intake module 136 may receive digital medical images, which may be used as an input for one or more trained machine learning systems. The target data may be received from any one or any combination of server systems 110, physician servers 121, hospital servers 122, clinical trial servers 123, research lab servers 124, and/or laboratory information systems 125. Target data may come from real sources (e.g., humans, animals, etc.) or may come from synthetic sources (e.g., graphics simulators, graphics rendering engines, 3D models, etc.). The target data intake module 136 may create or receive the one or more datasets of target data, e.g., digital medical images. For example, the datasets may include one or more datasets corresponding to digital medical images and/or, optionally, one or more datasets corresponding to clinical data. In some examples, a subset of target data may overlap between or among the various datasets for images and/or clinical data. The target datasets may be stored on a digital storage device, e.g., one of storages devices 109.

Population module 137 may include any suitable machine learning systems, including but not limited to, graph neural networks, convolutional neural networks, transformer neural networks, etc. Population module 137 may execute the various machine learning systems generated by training graph generation platform 131, e.g., training population prediction module 134, to facilitate the generation and/or population of a gene network graph. Population module 137 may populate the gene network graph with expression levels that are inferred based on one or more features identified from processing one or more medical images and, optionally, clinical data, and the associated expression levels learned for those one or more features.

The output interface 138 may be used to output the populated gene network graph (e.g., to a screen, monitor, storage device, web browser, etc.). According to some techniques, output interface 138 may output the populated gene network graph to graph prediction system 102 for use as input in a subsequent process described below. Populated gene network graphs and other data produced or used by graph generation system 101 may be stored in one or storage devices 109.

FIG. 1D depicts an exemplary populated gene network graph 150, e.g., as may be output by graph generation system 101 and/or graph prediction system 102. As depicted in FIG. 1D, gene network graph 150 may include one or more nodes that correspond to genes in the network, such as nodes for one or more transcription factors 152, nodes for one or more proteins 153, etc. The relationships, e.g., continuous expression levels, between the nodes may be represented by one or more edges. A visual representation of the edge within the gene network graph 150 may depict characteristics of the relationships, such as presence of genetic evidence, positive or negative effects, expression, and/or regulation, etc. For example, edge 154a may represent genetic evidence of protein 153 HOG1 inducing a negative effect on protein 153 CHS. In another example, edge 154b may represent transcription factor 152 P1F3 inducing a positive effect of expression on transcription factor 152 LHY. In another example, edge 154c may represent transcription factor 152 P1F3 inducing upregulation of promotor binding to protein 153 CAB1. Any other suitable nodes, edges, and/or combination of nodes and edges may be represented on gene network graph 150. While gene network graph 150 may include and depict populated nodes, it should be understood that gene network graphs, such as unpopulated gene network graphs received as input by graph generation system 101, may not include or depict populated nodes and the resultant signal pathways and/or relationships.

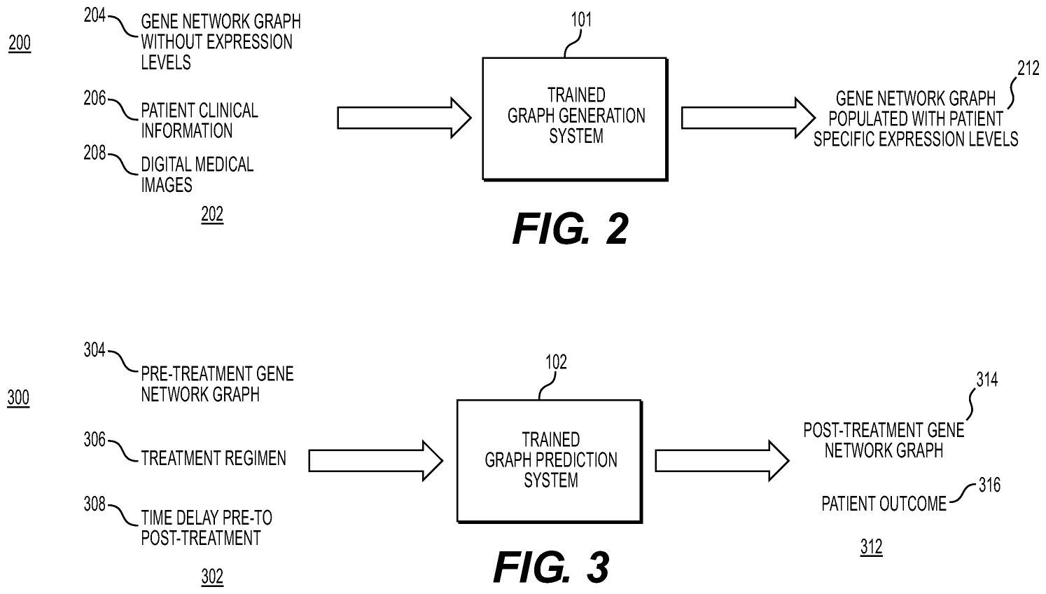

FIG. 2 depicts a schematic 200 of an exemplary system (e.g., graph generation system 101) implemented for generating populated gene network graphs. As depicted in FIG. 2, graph generation system 101 may receive one or more inputs 202, e.g., at target data intake module 136. The one or more inputs 202 may include, but are not limited to, one or more gene network graphs without expression levels 204, patient clinical information 206, digital medical images 208 of the patient, or any combination thereof. The one or more inputs 202 may be received from any one or any combination of server systems 110, physician servers 121, hospital servers 122, clinical trial servers 123, research lab servers 124, and/or laboratory information systems 125. The one or more inputs 202 may be processed by graph generation system 101 using one or more trained machine learning systems (e.g., population module 137) to output a gene network graph populated with patient-specific expression levels (populated gene network graph) 212. The outputted populated gene network graph 212 may be stored, e.g., in storage devices 109, or received by graph prediction system 102 for further processing.

FIG. 1C depicts an exemplary system (e.g., graph prediction system 102) for predicting behavior of gene network graphs, according to an exemplary technique of the present disclosure. The graph prediction system 102 may include a training graph prediction platform 141 and/or a target graph prediction platform 145.

According to one technique, the training graph prediction platform 141 may include software modules, such as a training data intake module 142 and a training prediction module 147. Training data intake module 142, according to one aspect, may create or receive training data that may be used to train one or more machine learning systems to generate post-treatment populated gene network graphs and/or predict treatment outcomes. The training data may be received from any one or any combination of server systems 110, physician servers 121, hospital servers 122, clinical trial servers 123, research lab servers 124, and/or laboratory information systems 125. Training data may come from real sources (e.g., humans, animals, etc.) or may come from synthetic sources (e.g., graphics simulators, graphics rendering engines, 3D models, etc.). The training data intake module 142 may create or receive the one or more datasets of training data. For example, the datasets may include one or more datasets corresponding to gene network graphs for a plurality of patients (e.g., pre-treatment and post-treatment gene network graphs), one or more datasets corresponding to treatment data for the plurality of patients (e.g., type of treatment, dosage, etc.), one or more datasets corresponding to pre-to-post-treatment time delay, etc. In another example, each dataset may be patient-specific and include a pre-treatment gene network graph, a post-treatment gene network graph, treatment data, and/or a time period between pre- and post-treatment gene network graphs for a given patient. In some examples, a subset of training data may overlap between or among the various datasets for gene network graphs, treatment data, and/or time delay data.

In some examples, the training data may be a direct output of one or more of the machine learning systems. In other examples, the output of one or more of the machine learning systems may be used as input to further processes that enable prediction of gene network graph changes. The training datasets may be stored on a digital storage device, e.g., one of storages devices 109.

The training prediction module 143 may generate, using the training data as input, one or more machine learning systems capable of predicting changes in the gene network graph in response to, e.g., a proposed treatment regimen. In some examples, a third party may generate the one or more trained machine learning systems and provide the trained machine learning system(s) to server systems 110 for storage (e.g., in storage devices 109) and/or execution by graph prediction system 102. Training prediction module 143 may train a transformer, a graph neural network, or any other suitable type of machine learning system to predict a post-treatment gene network graph that depicts, e.g., how gene expression levels may change from a pre-treatment gene network graph in response to a particular treatment. Training prediction module 143 may store post-treatment gene network graphs in a database, e.g., storage devices 109, along with other gene network graphs, e.g., pre-treatment gene network graphs and populated gene network graphs.

Additionally or alternatively, the training prediction module 143 may generate, using the training data as input, one or more machine learning systems capable of predicting treatment outcomes (also referred to herein as patient outcomes). In some examples, a machine learning system may be generated for each of the different tissue types, e.g., tumor types, to learn a corresponding tissue response to a given treatment. In other examples, one machine learning system may be generated that is capable of predicting treatment outcomes for more than one tissue type. Methods for training the one or more machine learning systems of training prediction module 143 are described below.

According to one technique, the target graph prediction platform 145 may include software modules, such as a target data intake module 146, a prediction module 147, and an output interface 148. Target data intake module 146 may receive one or more target inputs, including, but not limited to, a pre-treatment gene network graph, a treatment regimen, a pre-to-post-treatment time delay, etc. For example, the one or more target data may be received from any one or any combination of the server systems 110, physician servers 121, hospital servers 122, clinical trial servers 123, research lab servers 124, and/or laboratory information systems 125.

Target data intake module 146 may provide the one or more inputs to the prediction module 147 to predict changes in the gene network graph and/or treatment outcomes. The prediction module 147 may be comprised of one or more components, described in more detail below. Prediction module 147 may execute the various machine learning models generated by training graph prediction platform 141 to facilitate the prediction of gene network graph changes and/or treatment outcomes.

Prediction module 147, according to one aspect, may receive a request to predict one or more changes in a gene network graph and/or treatment outcomes, and execute one or more of the machine learning systems trained by training graph prediction platform 141 to predict the one or more changes to the gene network graph and/or treatment outcomes responsive to the request. For example, the request may be received from any one or any combination of the server systems 110, physician servers 121, hospital servers 122, clinical trial servers 123, research lab servers 124, and/or laboratory information systems 125. In another example, the request may be automatically generated by graph prediction system 102 in response to detecting an output from another system, e.g., from graph generation system 101. In some implementations, prediction module 147 may be configured to automatically predict one or more changes in a gene network graph and/or treatment outcomes in response to detecting a significant change or deviation in the populated gene network expression levels compared to a baseline, in response to detecting a change in a treatment (e.g., in drug dosing), etc.

Prediction module 147 may include any suitable machine learning systems, including but not limited to, graph neural networks, convolutional neural networks, transformer neural networks, etc. Prediction module 147 may execute the various machine learning systems generated by training graph prediction platform 141, e.g., training prediction module 143, to facilitate the generation and/or population of a post-treatment gene network graph and/or outcome data. Post-treatment gene network graph may depict a gene network graph with predicted expression values in response to a stimulus, e.g., time, a treatment regimen, etc. Outcome data may include clinical data, such as overall survival data, progression-free survival with corresponding censored data, drug treatment outcome data, time delay before treatment (e.g., between diagnosis and treatment), remission rate, etc.

The output interface 148 may be used to output the predicted post-treatment populated gene network graph and/or outcome data (e.g., to a screen, monitor, storage device, web browser, etc.).

FIG. 3 depicts a schematic 300 of an exemplary system (e.g., graph prediction system 102) implemented for predicting changes to a gene network graph. As depicted in FIG. 3, graph prediction system 102 may obtain one or more inputs 302, e.g., at target data intake module 146. As discussed herein, the one or more inputs 302 may be received from any one or any combination of the server systems 110, physician servers 121, hospital servers 122, clinical trial servers 123, research lab servers 124, and/or laboratory information systems 125. The one or more inputs 302 may include, but are not limited to, one or more pre-treatment gene network graphs 304, a treatment regimen 306, an optional pre-treatment-to-post-treatment time delay 308, etc. Pre-treatment gene network graph 304 may depict a gene network graph with expression values before the occurrence of a stimulus, e.g., a time delay, a treatment regimen, etc. Treatment regimen 306 may include the dosage, schedule, timing, etc. Pre-treatment-to-post-treatment time delay 308 may include the time between diagnosis and treatment. Using one or more trained machine learning systems described herein to process the inputs 302, graph prediction system 102 may provide, as one or more outputs 312, a post-treatment gene network graph 314 and/or a patient outcome 316. The outputs 312 may be associated with a time period indicated by the time delay 308.

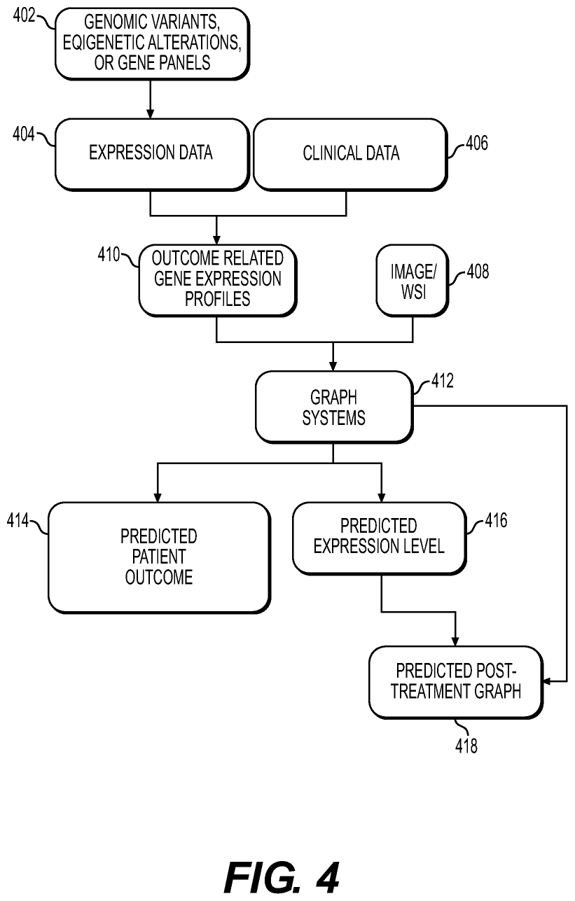

FIG. 4 depicts a flow diagram for an exemplary process for generating and predicting changes in gene network graphs, according to one or more techniques. As shown in FIG. 4, one or more inputs can be processed by one or more systems (e.g., graph systems 412) to generate one or more outputs. Graph systems 412 may include graph population system 101 and graph prediction system 102 operating in conjunction with one another. The one or more inputs may include genomics and/or epigenomic data 402 (e.g., genomic variants, epigenetic alterations, or gene panels), expression data 404 (e.g., RNA sequences and/or microarrays of genes), clinical data 406 (e.g., survival data and/or treatment responses), and/or medical images 408 (e.g., matched images or whole slide images (WSIs)).

Genomics and/or epigenomic data 402 may include genomic and epigenetic variants, e.g. point mutations, copy number variants, structural variants, histone modification, and/or hypermethylation. The genomics and/or epigenomic data 402, if available, may be used to identify corresponding gene expression profiles induced by genomic or epigenetic variants.

Expression data 404, e.g., RNA sequence expression levels, may include patient gene matrices across multiple cancer types (pan-cancer), patient gene matrices across a single cancer type, patient gene matrices at various stages of treatment, such as at different time stamps (e.g. time series), patient gene matrices after various combinatorial therapies, etc. In some examples, the values may be a normalized expression level, e.g. Normalized FPKM values (Fragments Per Kilobase of transcript per Million mapped read), or Microarray gene data. Genes with flat gene expression profiles may be excluded. In some embodiments, expression data 404 may be in the form of a populated gene network graph that may include expression data. Expression data 404 may be used to identify survival-related genes or drug response-related genes using Univariate and/or Multivariate Cox proportional hazards (Cox PH) regression.

The clinical data 406 may include age, medical history, cancer treatment history, family history, past biopsy or cytology information, tumor sequencing information, mRNA expression levels, gene network graphs (pre-treatment and/or post-treatment), overall survival data, progression-free survival with corresponding censored data, drug treatment outcome data, etc. As discussed herein, graph generation system 101 may be optionally trained using particular types of clinical data 406 (e.g., cancer treatment history, family history, mRNA expression levels, etc.) while graph generation system may be trained using other types of clinical data 406 (e.g., overall survival data, progression-free survival with corresponding censored data, drug treatment outcome data, etc.). The medical images 408 may be patient-to-sample level. In some examples, an image or WSI may be divided into multiple tiles for analysis and/or processing. However, a predicted expression level may be based on aggregated values from multiple tiles.

Gene expression profiles 410 may include pre- and/or post-treatment gene network graphs. Gene expression profiles 410 may be based on expression data 404 and/or at least a portion of clinical data 406 associated with treatment and outcome data, and thus gene expression profiles 410 may be outcome related. Gene expression profiles 410 may comprise a continuous value vector to indicate the normalized expression levels of a list of genes, or an integer vector (e.g., of 1, 0, −1) for a list of genes to indicate if a gene may be over-expressed, unchanged, or down-regulated. Based on the predicted gene expression profile 410, the genes that are highly expressed or down-regulated may be identified. Pathway enrichment analysis may be used to determine the associated pathways, e.g., T-cell receptor, DNA repair pathways etc., and relate the pathways to potential treatment therapies.

The gene expression profiles 410 may be used to train machine learning systems executed by graph systems 412 to infer changes in gene expression (e.g., changes in gene graph networks) and/or patient outcomes given a particular treatment regimen and/or time delay, e.g., using Bayesian inference, correlation inference, Boolean inference algorithms or other suitable models, for different sample subsets. For example, possible or likely treatment outcomes for a patient and/or prognosis, e.g., good or poor, for a patient may be inferred based on how a gene network graph (e.g., an oncogenic signaling pathway) for the patient is predicted to change based on the treatment received. This process may help identify gene interactions that may contribute to high risk of cancers or drug resistance and/or provide insights on different clinical outcomes for the same mutant gene, e.g., identify HER2+ patients who do not have good response to anti-HER2+ drugs (e.g., lapatinib and trastuzumab) and anti-HER2+ combinatorial drug therapies.

As discussed herein, an identified subset of the inputs may be used to train one or more machine learning systems of graph systems 412, which may include graph generation system 101 and/or graph prediction system 102. For example, genomics and/or epigenomic data 402, clinical data 406, and/or medical images 408 may be used to train graph generation system 101. In another example, expression data 404, clinical data 406, and/or gene expression profiles 410 may be used to train graph prediction system 102.

In some techniques, the graph systems 412 may generate one or more outputs. For example, the graph systems 412 may output one or more predicted a patient outcome 414, predicted expression levels 416, and/or a predicted post-treatment graph 418 (e.g., based on predicted expression levels 416). In some examples, the predicted patient outcome 414 may include a heat map of affected areas and/or histological patterns associated with expression levels, including a gene-by-gene matrix with values (e.g., 1, 0, and/or −1 for up-regulation, no interaction, or down-regulation (i.e. inhibition), respectively), or it can be a matrix of signed values (weights), the value indicating the association between genes (i.e. how the expression of one gene changes the expression of other genes). The predicted expression levels 416, e.g., the predictions including expression levels at future time points, may include different stage/phases of drug treatment (with or without varied dosages), which may be used to infer predicted post-treatment graph 418 and/or a response to treatment. In some examples, graph systems 412 may use one or more inputs, e.g., expression data 404 and/or medical images 408, to infer predicted post-treatment graph 418 directly.

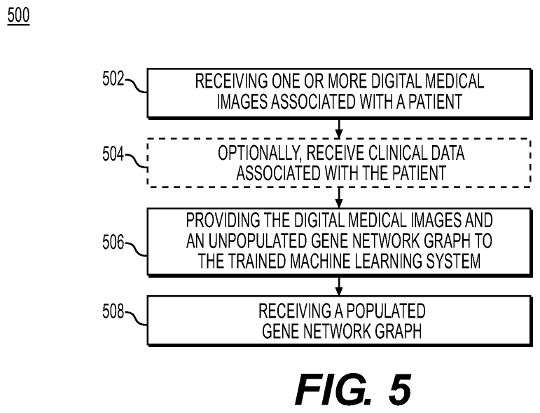

FIG. 5 depicts a flow chart of an exemplary method 500 for populating gene network graphs, according to one or more techniques. At step 502, the system, e.g., graph generation system 101, may receive one or more digital medical images associated with a patient. Optionally, at step 504, clinical data associated with the patient may also be received. The input data may be generated and/or stored by the systems described herein, e.g., storage devices 109, or received from one or more of physician servers 121, hospital servers 122, clinical trial servers 123, research lab servers 124, and/or laboratory information systems 125.

At step 506, the one or more digital medical images and an unpopulated gene network graph associated with the patient may be provided to the trained machine learning system. If the clinical data is optionally received, the clinical data may also be provided to the trained machine learning system. The trained machine learning system may process the one or more digital medical images (and optionally the clinical data) to output at least one populated gene network graph. The populated gene network graph may be received from the trained machine learning system at step 508. For example, for a patient with a given cancer genotype, graph generation system 101 may use a multi-modal deep neural network to predict the patient's populated gene network graph based the patient's digital medical images, e.g., digital whole slide images of cancerous tissue.

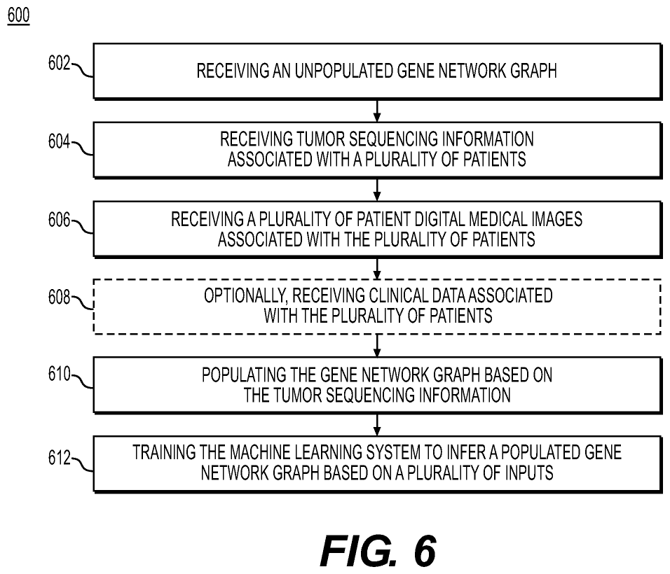

The machine learning system may be trained as described in FIG. 6. The machine learning system may be trained to infer a patient-specific populated gene network graph based on one or more inputs, e.g., mRNA and/or tumor sequencing data, patient clinical information, digital medical images, etc.

FIG. 6 depicts an exemplary method 600 for training a machine learning model to be implemented by graph generation system 101, according to one or more techniques. The machine learning model may be trained to infer a populated gene network graph based at least on digital medical images. Exemplary method 600 (e.g., steps 602-610) may be performed by graph generation system 101. Exemplary method 600 may include one or more of the following steps.

At step 602, an unpopulated gene network graph may be received. An unpopulated gene network graph may depict general relationships between genes, proteins, mRNA, etc., but may not include any gene expression levels. For example, an unpopulated gene network graph may depict that a relationship exists between individual genes of the SWI/SNF complex (e.g., the ARID1A, ARID1B, ARID2, PBRM1, SMARCA4 and SMARCB1 genes), but not how those genes may interact in a particular individual's cells. In other words, general gene relationships may be depicted using an unpopulated gene network graph, but a patient's particular amount or degree of gene interactions (e.g., whether certain gene interactions for a given patient happen to a higher or lower degree) are not indicated due to lack of gene expression levels. In some examples, the general relationships between genes, proteins, mRNA, etc. depicted in an unpopulated gene network graph may be received from a public database that may store a collection of general relationship data for various sets of genes.

At step 604, tumor sequencing information associated with a plurality of patients may be received. The tumor sequencing information may comprise patient-specific gene sequences (e.g., driver regions, promotor regions, exons, etc.), variation data based on a relevant patient population (e.g., gene variations associated with Ashkenazi Jewish populations), etc. The tumor sequencing information may indicate expression levels associated with genes represented by nodes in the unpopulated gene network graph. At step 606, the machine learning system may receive a plurality of patient digital medical images associated with the plurality of patients. As discussed herein, the digital medical images may be any suitable composition, e.g., digital multiplex immunofluorescent images, digital multiplex immunohistochemistry images, magnetic resonance imaging (MRI), computed tomography (CT), X-ray, nuclear medicine imaging, ultrasound, etc. Optionally, at step 608, clinical data associated with the plurality of patients may be received for training. Clinical data may include cancer treatment history, family history, mRNA expression levels, etc. Steps 602, 604, 606, and 608 may be performed simultaneously and/or separately.

At step 610, for one or more of the plurality of patients, the unpopulated gene network graph may be populated based on tumor sequencing information of the respective patient. In other words, training population module 133 may correlate tumor sequencing data with expression level data to generate a populated gene network graph that includes expression levels for the genes depicted by nodes of the gene network graph. Populating the gene network graph may further comprise determining whether there are missing expression level values in the gene network graph and using label propagation techniques to infer missing values. As discussed herein, tumor sequencing data may not provide or include expression levels for all genes. In such instances, expression level values for genes that are missing may be determined using, e.g., directed label propagation techniques, as discussed herein.

At step 612, the machine learning system may be trained using a plurality of inputs, such as the unpopulated gene network graph, the digital medical images and/or the clinical data, if optionally received. The machine learning system may be trained to infer a populated gene network graph for a patient based on one or more medical images of the patient using supervised or semi-supervised learning. The trained machine learning system may be output to digital storage, e.g., storage devices 109.

In some examples, the supervised machine learning system may be trained using classification or regression. In such examples, the supervised machine learning system may include a multi-modal deep neural network, graph neural networks, transformer neural networks, convolutional neural network (CNN), a recurrent neural network (RNN), or a multi-layer perceptron (MLP), among other similar examples. To enable learning, a digital medical image may be provided as input to the machine learning system. The machine learning system may then output predicted gene sequencing data that may be used to populate a gene network graph. The predicted gene sequencing data may be compared to corresponding gene sequencing data to determine a loss or error, which can be used to update the parameters of the machine learning system to reduce the loss or error. The corresponding gene sequencing data may be a portion of the training gene sequencing data that corresponds to the digital medical image and indicates known cancerous tissue aspects and/or genotypes in the digital medical images. The machine learning system may be modified or altered (e.g., weights and/or bias associated with one or more nodes and/or layers may be adjusted) based on the error to improve an accuracy of the machine learning system. This process may be repeated for each of the training digital medical images received or at least until a determined loss or error is below a predefined threshold. In some examples, a portion of the training images may be withheld and used to further validate or test the machine learning system.

In some examples, the machine learning model may include a Sequence to Sequence (“Seq2Seq”) model, e.g., a transformer Seq2Seq model. The transformer Seq2Seq model may include an encoder model and a decoder model. The transformer Seq2Seq model may be configured to, for example, receive as input tiles from the digital medical images and/or vector embeddings of the tiles (and optional clinical data), which the encoder model encodes and/or compresses. The decoder model may receive and decode the encoded and/or compressed vector embeddings from the encoder model to output a populated gene network graph. The decoder output may be compared to an actual populated network graph for the patient (e.g., that was populated using tumor sequencing information for the patient) to determine a loss or error, which may be used to update the parameters of the machine learning system to reduce the loss or error. In some examples, the transformer Seq2Seq model may receive a variable amount of data as input, and produce a fixed-size populated gene network graph.

In some aspects, the populated gene network graph outputted by the trained machine learning system of graph generation system 101 may be a pre-treatment gene network graph (e.g., a gene network graph depicting gene expression levels before the patient has undergone treatment). Using this graph as an input, the graph prediction system 102 may be configured to predict an outcome and/or a post-treatment gene network graph based on a proposed treatment.

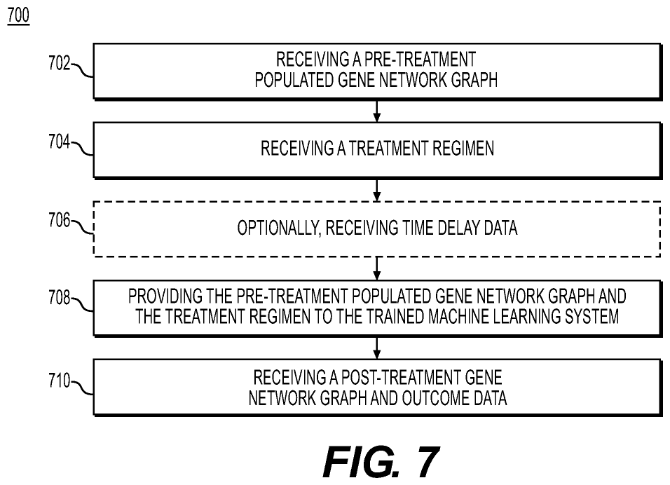

FIG. 7 depicts a flow chart of an exemplary method 700 for predicting gene network graph changes, e.g., in response to a proposed treatment regimen, according to one or more techniques. Exemplary method 700 (e.g., steps 702-710) may be performed by graph prediction system 102. Exemplary method 700 may include one or more of the following steps.

At step 702, a pre-treatment populated gene network graph may be received. As discussed herein, the pre-treatment populated gene network graph may comprise a gene network graph before a patient receives any treatment, a gene network graph after a patient has received an initial treatment, etc. For example, for a patient that has received an initial treatment, the pre-treatment populated gene network graph may be populated based on gene expression values subsequent to the first treatment but prior to a proposed second treatment. In some examples, the pre-treatment populated gene network graph may be generated by the graph prediction system 101.

At step 704, one or more proposed treatment regimens may be received. The proposed treatment regimens may include monotherapy treatments (e.g., chemotherapy, MET inhibitors, etc.), combinatorial treatments (e.g., cisplatin and Taxol for the treatment of pancreatic cancer), timing data (e.g., frequency of treatment, duration of treatment, etc.), dosage data (e.g., amount per dose, amount of total doses), etc. In either or both of steps 702 and 704, the inputs may be received at graph prediction system 102, e.g., at target graph prediction platform 145, and/or stored, e.g., by storage devices 109. In some examples, the proposed treatment regimen may be vectorized into a vector embedding. Optionally, at step 706, time delay data may be received. The time delay data may define a period of time having passed between the pre-treatment populated gene network graph received at step 702 and the post-treatment gene network graph and/or outcome to be predicted by the trained machine learning system. For example, if a user is interested in determining how a patient's gene expression levels will change based on the proposed treatment regimens after a period of one year, the time delay data may indicate one year.

At step 708, the pre-treatment populated gene network graph, one or more proposed treatment regimens may be provided as input data to the trained machine learning system, e.g., target graph prediction platform 145. Optionally, the input data may also include time delay data, if received. The trained machine learning system may process the input data to output at least one post-treatment populated gene network graph and/or outcome data at step 710. The post-treatment populated gene network graph may have the same structure or topology as the pre-populated gene network graph (e.g. the same nodes and edges), but may have differing values associated with one or more of the nodes indicating a change in expression levels (e.g., a change in behavior) for the genes represented by the nodes. If the optional time delay data is received, the post-treatment populated gene network graph and/or outcome data predicted and output by the trained machine learning system may be at the given time delay (e.g., at the defined time period following the pre-treatment graph). Alternatively, if no time delay data is received, a set of post-treatment populated gene network graphs and/or outcome data at a series of time delays may be predicted and output by the trained machine learning system. For example, the series of time delays may be pre-defined intervals (e.g., every 6 months, every year, every 3 years, etc.) following the pre-treatment graph. Outcome data may include, e.g., predicted treatment success rate, predicted gene interactions, predicted survival rate, risk of metastases, T-cell receptor immunotherapy resistance, etc. Any of the inputs to graph prediction system 102 (e.g., pre-treatment populated gene network graphs), the outputs from graph prediction system 102 (e.g., post-treatment populated gene network graphs), and/or any other data may be stored, e.g., in storage devices 109.

In one example, the post-treatment populated gene network graph may inform the predicted efficacy and/or chemoresistance of a proposed treatment regimen. In another example, for a patient with a given acute lymphoblastic leukemia genotype, graph prediction system 102 may use a transformer to predict the patient's post-treatment populated gene network graph based the pre-treatment populated gene network graph and the proposed treatment of azacitidine. In another example, graph prediction system 102 may use a graph neural network to predict whether a patient may develop endometrial hyperplasia, an overgrowth of normal cells, or atypical endometrial hyperplasia, an overgrowth of abnormal cells, based on changes in the pathways at different time points.

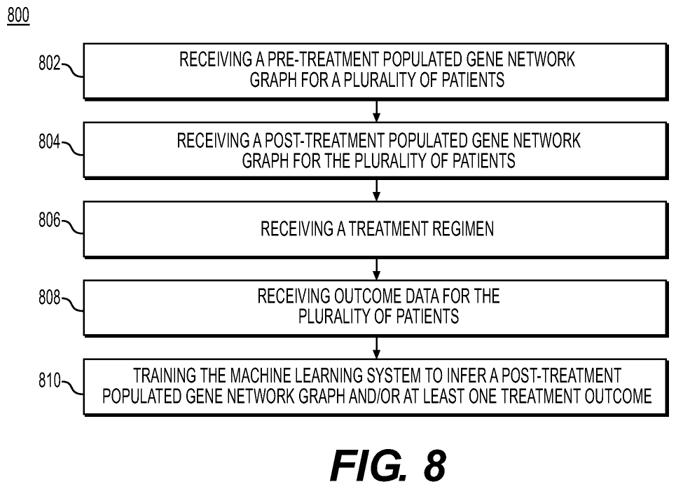

The machine learning system may be trained as described in FIG. 8. Exemplary method 800 (e.g., steps 802-810) may be performed by training graph prediction platform 141 of graph prediction system 102. Exemplary method 800 may include one or more of the following steps.

A pre-treatment populated gene network graph for a plurality of patients and a post-treatment populated gene network graph for the plurality of patients may be received at step 802 and 804, respectively. The post-treatment populated gene network graph may have been generated at a given time period (e.g., a given time delay) after the pre-treatment populated gene network graph. In some examples, a given patient may have more than one post-treatment populated gene network graph at different time delays following the pre-treatment populated gene network graph. Time delay data indicating the time period having passed between the pre-treatment populated gene network graph and the one or more post-treatment populated gene network graph may be received for one or more of the plurality of patients for use in training. At step 806, the machine learning system, e.g., the training graph prediction platform 141, may receive a treatment regimen undergone by the plurality of patients. As discussed herein, the treatment regimen may include types of treatments (e.g., monotherapy treatments, combinatorial treatments), timing data, dosage data, etc. Timing data may include treatment time delay data, e.g., the time elapsed from diagnosis to beginning treatment. A vector embedding may be generated to describe or represent the treatment regimen.

At step 808, the machine learning system, e.g., the training graph prediction platform 141, may receive outcome data for the plurality of patients. The outcome data may include clinical data, such as overall patient survival rates, progression-free survival rates, Response Evaluation Criteria in Solid Tumors (RECIST), pathologic complete response data, or drug treatment outcomes, among other similar data.

In some instances, any or all of the pre-treatment populated gene network graph, the post-treatment gene network graph, the proposed treatment regimen, and/or the outcome data may be vectorized into vector form. The vector forms of the respective inputs may be received by the machine learning system. Steps 802, 804, 806 and 808 may be performed simultaneously and/or separately.

At step 810, the machine learning system may be trained to infer a post-treatment populated gene network graph and/or at least one treatment outcome. The machine learning system may be trained using one or more of the inputs from steps 802-808. The machine learning system may use any known methods for training, e.g., supervised learning. The trained system may be output to digital storage, e.g., storage devices 109.

In some examples, the supervised machine learning system may be trained using strong annotations (e.g. known patient outcomes and/or known changes in gene expression from the pre-treatment network graph and the post-treatment network graph in response to a given treatment). In such examples, the supervised machine learning system may include a graph neural network, a transformer neural network, a convolutional neural network (CNN), or a multi-layer perceptron (MLP), among other similar examples. To enable learning, pre-treatment gene sequencing data (e.g., in form of a pre-treatment network graph) for a patient, post-treatment gene sequencing data (e.g., in form of a post-treatment network graph) for a patient, a corresponding treatment regimen undergone by the patient, and outcome data may be provided as input to the machine learning system. Optionally, time delay data indicating a time period between the pre-treatment gene network graph and the post-treatment gene network graph to be predicted that is, for example, equal to the time period between the actual pre- and post-treatment gene network graph for the patient may also be provided as input. The machine learning system may then output a predicted post-treatment gene sequencing data that may be used to populate a post-treatment gene network graph and/or a predicted patient outcome (e.g., at the given time delay if optionally received). The predicted post-treatment gene network graph may be compared to the actual post-treatment gene network graph for the patient to determine a loss or error. Similarly, the predicted patient outcome may be compared to the actual patient outcome (e.g., obtained from the clinical data). The actual post-treatment gene network graph and patient outcome may be a portion of a strong annotation of the training gene sequencing data that corresponds to the proposed treatment regimen and indicates changes in known gene expression (e.g., decreased methylation of a driver gene in response to a given treatment) and/or outcome data from the pre-treatment gene network graph to post-treatment gene network graph. The machine learning system may be modified or altered (e.g., weights and/or bias associated with one or more nodes and/or layers may be adjusted) based on the error to improve an accuracy of the machine learning system. This process may be repeated for each of the training proposed treatment regimens received or at least until a determined loss or error is below a predefined threshold. In some examples, a portion of the training treatment regimens may be withheld and used to further validate or test the machine learning system.

### Example Application: Surrogate of Treatment Outcome

Clinical outcomes directly measure whether patients in a trial feel or function better, or live longer. The benefit or likely benefit of a therapy, as measured by clinical outcomes, may be assessed to determine whether it outweighs any adverse effects. Surrogate endpoints may be used instead of clinical outcomes in some clinical trials, when the clinical outcomes may take a long time to study.

Aspects disclosed herein can be used to identify oncogenic signaling pathways, the activation or inhibition of which are associated with or can predict the outcomes (e.g., predict responses to drugs, overall survivals, progression free survivals, etc.) as appropriate surrogate endpoints. This can support clinical trial design by identifying patients who are more likely to respond to the drug based on the signaling pathways that the drug targets, despite not having outcome data for the drug. This is helpful especially at the early stage of clinical trial design where preliminary outcome data is not available.

### Example Application: Biomarker Screening and Development

Screening a biomarker derived from a single genomic variant can fail due to the limited number of positive cases. In contrast, screening a signaling pathway derived from multiple genes, increases the sample size, and may enable screening of rare variants and/or rare tumor types. For example, the prevalence of mutation in each of the individual genes of the SWI/SNF complex (ARID1A, ARID1B, ARID2, PBRM1, SMARCA4 and SMARCB1) may be low in some tumors, while the prevalence of mutations in the complex is collectively found in approximately 20% of all tumors.

Aspects disclosed herein can be used to identify signaling pathways as pharmacodynamic biomarkers, as well as predictive biomarkers to single and combined therapies when often each drug targets a different gene but the same pathway. Driver genetic and epigenetic variants associated with genes contributing to the functional disruption of the same signaling pathways or networks may be integrated, which helps to increase the number of positive cases and screen biomarkers at the functional signaling pathway or complex level.

### Example Application: Identify Rare Tumor Subtypes

Gene expression assays may be used for tumor classification. Tumor samples may be clustered based on gene expression profile, in which retrospective analysis identifies clinical implication of each tumor subtype. For example, PAM50 gene expression assay helps to reveal intrinsic subtypes of breast tumors, e.g., Luminal A, Luminal B, Basal-like, and Normal subtypes of breast cancers. However, with a varied number of genes in an assay, the tumor subtypes might vary, and it may also be difficult to identify rare tumor subtypes based a limited set of genes.

Aspects disclosed herein can be used to detect histologic feature related oncogenic signaling pathways, where the detection of pathways may be used as a complement to or replacement for gene expression assay to help patient stratification. The tumor samples may be clustered based on the computer learned histologic patterns associated with activation of oncogenic pathways. The retrospective analysis along with clinical information may be used to identify rare tumor subtypes and the associated signaling pathways or a gene complex, providing guidance on evaluating treatment strategies on different risk groups of patients.

### Example Application: Estimate Risk of Distant Metastases

Metastasis is the key cause of failure of cancer therapy and mortality. Adjuvant chemotherapy is often used for distant control. However, not all patients can benefit from adjuvant chemotherapy, and particularly, some patients may even get worse outcomes after the treatment. Evaluation of the risk of distant metastasis and identifying patients who may benefit from adjuvant chemotherapy for distant control facilitates treatment planning. There are specific mutations, and signaling processes that may contribute to metastasis. As one example, Ras mutations present in about 50% of metastatic tumors, and Ras protein activates multiple downstream signaling pathways. As another example, epithelial-mesenchymal transition (EMT), which is a spectrum of transitional stages between the epithelial and mesenchymal phenotypes, enable cells to gain a migratory phenotype, as well as induce multiple immunosuppression, drug resistance, evasion of apoptosis mechanisms.

Aspects disclosed herein may be used to detect signaling pathways that regulate progression and promote the acquisition of the metastatic phenotype. For example, the above-described systems and methods may be used identify the histological pattern associated with Ras mutations and downstream signaling pathways, and predict the activation of Ras signaling pathways to infer the risk of distant metastases. Additionally, the above-described systems and methods may be used to link the learned histologic features with epithelial phenotype versus mesenchymal phenotype, which detect the transformation type whether it is transforming from epithelial to mesenchymal (EMT) or from mesenchymal to epithelial (MET). Along with prognosis data, these identifications and detections help to predict the risk of distant metastasis after surgery and help to identify if patients would be benefit from adjuvant chemotherapy.

### Example Application: Evaluation of Therapeutic Intervention and Synthetic Lethality

Synthetic lethality is an approach to target cancer cells harboring specific, undruggable cancer mutations, which is a type of genetic interaction perturbing multiple genes simultaneously that results in cell death. Synthetic lethal screens have the potential to identify new vulnerabilities incurred by specific cancer mutations for developing new therapeutics. Synthetic lethal effects at the pathway level are more reproducible than at the gene level.

Aspects disclosed herein may be used to identify the visual pattern associated with oncogenic signaling pathways, e.g., DNA damage response pathways, under normal and disease status (functional disruption). By detecting the difference in signaling pathways before and after a therapeutic intervention, efficacy of treatment may be estimated and chemoresistance may be further predicted. Additionally, such identification and detection may facilitate the screening of synthetic lethality strategies for developing more effective targeted drugs in cancer therapy.

### Example Application: Predict Resistance to T-Cell Receptor (TCR)-Based Immunotherapy

TCR-based immunotherapies may have potential use for the treatment of patients with diverse solid cancers. However, there are multiple pathways associated with TCR resistance, e.g., loss of function, loss of heterozygosity and epigenetic silencing of key genes involved in antigen processing, presentation, and the interferon response pathways.

Aspects disclosed herein may be used to identify the histological pattern associated with TCR resistance (e.g., T cell-receptor signaling pathways or B-cell receptor signaling pathways), and evaluate the effectiveness of TCR-based treatments combined with adjunctive immunotherapies, including co-infusion with immune checkpoint inhibitors and immune-stimulating cytokines in overcoming the treatment resistance.

### Example Application: Predict Tumor Progression from Benign to Malignant

Oncogenic drivers are found in a range of benign conditions as well as in normal tissues. Many factors may induce the transformation from benign to malignant state, including tissue microenvironment, genomic driver co-factors or co-loss of tumor suppressors, the size of mutant clones, etc. For example, atypical endometrial hyperplasia is a precancerous condition that can develop in the lining of the uterus. It is an overgrowth of abnormal cells, or it can develop from endometrial hyperplasia, which is an overgrowth of normal cells. Patients with atypical endometrial hyperplasia have a very high risk to develop endometrial cancer.

Aspects disclosed herein may be used to identify the histological patterns associated with genomic or epigenetic variants in normal samples, atypical endometrial hyperplasia, and endometrial tumor samples. For example, changes in pathways at different time points may be established to profile tumor progression. Additionally, minimum changes required in specific oncogenic signaling pathways to trigger the transformation from benign to malignant may be determined.

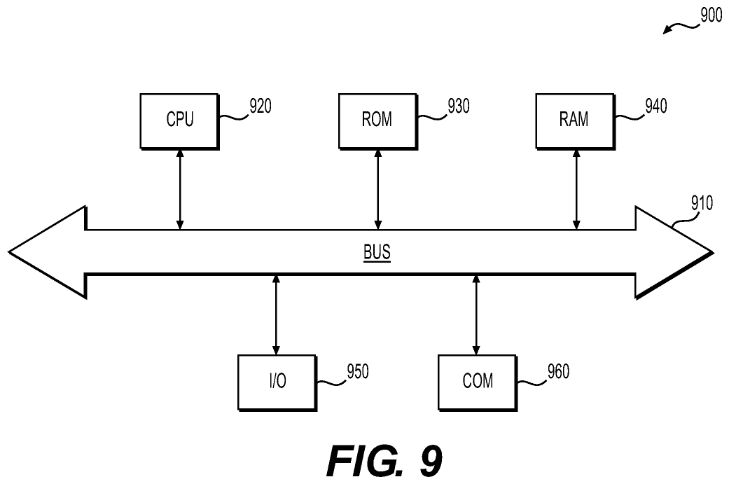

FIG. 9 illustrates an example system or device 900 that may execute techniques presented herein. Device 900 may include a central processing unit (CPU) 920. CPU 920 may be any type of processor device including, for example, any type of special purpose or a general-purpose microprocessor device. As will be appreciated by persons skilled in the relevant art, CPU 920 also may be a single processor in a multi-core/multiprocessor system, such system operating alone, or in a cluster of computing devices operating in a cluster or server farm. CPU 920 may be connected to a data communication infrastructure 910, for example a bus, message queue, network, or multi-core message-passing scheme.

Device 900 may also include a main memory 940, for example, random access memory (RAM), and also may include a secondary memory 930. Secondary memory 930, e.g. a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 930 may include similar means for allowing computer programs or other instructions to be loaded into device 900. Examples of such means may include a program cartridge and cartridge interface (such as that found in video game devices), a removable memory chip (such as an EPROM or PROM) and associated socket, and other removable storage units and interfaces, which allow software and data to be transferred from a removable storage unit to device 900.

Device 900 also may include a communications interface (COM) 960. Communications interface 960 allows software and data to be transferred between device 900 and external devices. Communications interface 960 may include a modem, a network interface (such as an Ethernet card), a communications port, a PCMCIA slot and card, or the like. Software and data transferred via communications interface 960 may be in the form of signals, which may be electronic, electromagnetic, optical or other signals capable of being received by communications interface 960. These signals may be provided to communications interface 960 via a communications path of device 900, which may be implemented using, for example, wire or cable, fiber optics, a phone line, a cellular phone link, an RF link or other communications channels.

The hardware elements, operating systems, and programming languages of such equipment are conventional in nature, and it is presumed that those skilled in the art are adequately familiar therewith. Device 900 may also include input and output ports 950 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Throughout this disclosure, references to components or modules generally refer to items that logically may be grouped together to perform a function or group of related functions. Like reference numerals are generally intended to refer to the same or similar components. Components and/or modules may be implemented in software, hardware, or a combination of software and/or hardware.

The tools, modules, and/or functions described above may be performed by one or more processors. “Storage” type media may include any or all of the tangible memory of the computers, processors or the like, or associated modules thereof, such as various semiconductor memories, tape drives, disk drives and the like, which may provide non-transitory storage at any time for software programming.

Software may be communicated through the Internet, a cloud service provider, or other telecommunication networks. For example, communications may enable loading software from one computer or processor into another. As used herein, unless restricted to non-transitory, tangible “storage” media, terms such as computer or machine “readable medium” refer to any medium that participates in providing instructions to a processor for execution.

The foregoing general description is exemplary and explanatory only, and not restrictive of the disclosure. Other embodiments may be apparent to those skilled in the art from consideration of the specification and practice of the invention disclosed herein. It is intended that the specification and examples be considered as exemplary only.

What is claimed is:

1. A method for processing digital medical images to populate gene network graphs, comprising:
receiving one or more digital medical images associated with a patient;
providing an unpopulated gene network graph and the one or more digital medical images as input to a trained machine learning system that is trained to populate the gene network graph with gene expression levels specific to the patient based on the one or more digital medical images; and
receiving, as output from the trained machine learning system, the gene network graph populated with the gene expression levels specific to the patient.

2. The method of claim 1, further comprising:
receiving clinical data associated with the patient; and
providing the clinical data as additional input to the trained machine learning system.

3. The method of claim 1, wherein the digital medical images include digital whole slide images, digital multiplex immunofluorescent images, or digital multiplex immunohistochemistry images.

4. The method of claim 1, wherein the machine learning system is trained by:
receiving, as training data, a plurality of digital medical images associated with a plurality of patients and populated gene network graphs for the plurality of patients; and
training the machine learning system, using the training data, to infer one or more of the populated gene network graphs based on the respective one or more digital medical images.

5. The method of claim 4, wherein the training data further includes clinical data associated with a plurality of patients, and the clinical data includes one or more of age, medical history, cancer treatment history, family history, past biopsy or cytology information, tumor sequencing information, mRNA expression levels.

6. The method of claim 4, wherein the gene network graphs for the plurality of patients are populated by:
receiving an unpopulated gene network graph, the unpopulated gene network graph comprising a gene network graph without expression levels;
receiving tumor sequencing information associated with the plurality of patients; and
populating the gene network graphs with expression levels for the plurality of patients based on the respective tumor sequencing information.

7. The method of claim 6, wherein the population of the gene network graphs for the plurality of patients comprises:
determining whether there are missing values for expression levels in the each of the populated gene network graphs; and
upon determining that there are missing values in one or more of the populated gene network graphs, using one or more label propagation techniques to infer the missing values.

8. The method of claim 7, wherein the one or more label propagation techniques include directed label propagation.

9. A method for training a machine learning system to populate a gene network graph, comprising:
receiving an unpopulated gene network graph, the unpopulated gene network graph comprising a gene network graph without expression levels;
receiving tumor sequencing information associated with each of a plurality of patients;
receiving one or more digital medical images associated with each of the plurality of patients;
populating, for each of the plurality of patients, the gene network graph to include expression levels based on the respective tumor sequencing information; and
training the machine learning system to infer one or more of the populated gene network graphs based on the respective one or more digital medical images.

10. The method of claim 9, wherein the digital medical images comprise digital whole slide images, digital multiplex immunofluorescent images, or digital multiplex immunohistochemistry images.

11. The method of claim 9, further comprising:
determining, for each populated gene network graph, whether there are missing values for expression levels in the gene network graph; and
upon determining that there are missing values, using one or more label propagation techniques to infer the missing values.

12. The method of claim 11, wherein the one or more label propagation techniques include directed label propagation.

13. The method of claim 9, further comprising:
receiving clinical data associated with each of the plurality of patients, wherein the machine learning system is further trained to infer the one or more populated gene network graphs based on the respective clinical data, the clinical data including age, medical history, cancer treatment history, family history, past biopsy or cytology information, tumor sequencing information, mRNA expression levels, or any combination thereof.

14. A system for processing digital medical images to populate gene network graphs, the system comprising:
at least one memory storing instructions; and
at least one processor configured to execute the instructions to perform operations comprising:
receiving one or more digital medical images associated with a patient;
providing an unpopulated gene network graph and the one or more digital medical images as input to a trained machine learning system that is trained to populate the gene network graph with gene expression levels specific to the patient based on the one or more digital medical images; and
receiving, as output from the trained machine learning system, the gene network graph populated with the gene expression levels specific to the patient.

15. The system of claim 14, further comprising:
receiving clinical data associated with the patient; and
providing the clinical data as additional input to the trained machine learning system.

16. The system of claim 14, wherein the digital medical images comprise digital whole slide images, digital multiplex immunofluorescent images, or digital multiplex immunohistochemistry images.

17. The system of claim 14, wherein the machine learning system is trained by:
receiving, as training data, a plurality of digital medical images associated with a plurality of patients and populated gene network graphs for the plurality of patients; and
training the machine learning system, using the training data, to infer one or more of the populated gene network graphs based on the respective one or more digital medical images.

18. The method of claim 17, wherein the training data further includes clinical data associated with a plurality of patients, and the clinical data includes one or more of age, medical history, cancer treatment history, family history, past biopsy or cytology information, tumor sequencing information, mRNA expression levels.

19. The method of claim 18, wherein the population of the gene network graphs for the plurality of patients comprises:
determining whether there are missing values for expression levels in the each of the populated gene network graphs; and
upon determining that there are missing values in one or more of the populated gene network graphs, using one or more label propagation techniques to infer the missing values.

20. The method of claim 19, wherein the one or more label propagation techniques include directed label propagation.
